(19) United States
(12) Patent Application Publication
(10) Pub. No.: US 2012/0009298 A1
(43) Pub. Date: Jan. 12, 2012

(54) METHOD FOR PRODUCING NOODLE, AND ENZYME PREPARATION FOR MODIFYING NOODLE

(75) Inventor: Noriaki YAMADA, Kawasaki-shi (JP)

(73) Assignee: AJINOMOTO CO., INC., Tokyo (JP)

(21) Appl. No.: 13/180,587

(22) Filed: Jul. 12, 2011

Related U.S. Application Data
(63) Continuation of application No. PCT/JP10/51908, filed on Feb. 3, 2010.

(30) Foreign Application Priority Data
Feb. 4, 2009 (JP) 2009-023298

(51) Int. Cl. A21D 8/04; C12N 9/04

(52) U.S. Cl. 426/10; 435/190

(57) ABSTRACT

A noodle having improved physical properties and improved taste may be obtained with an enzyme preparation for modifying a noodle, which contains α-glucosidase and glucose oxidase.

### CROSS REFERENCES TO RELATED APPLICATIONS

This application is a continuation of International Patent Application No. PCT/JP2010/051908, filed on Feb. 3, 2010, and claims priority to Japanese Patent Application No. 023298/2009, filed on Feb. 4, 2009, both of which are incorporated herein by reference in their entireties.

### BACKGROUND OF THE INVENTION

1. Field of the Invention

The present invention relates to methods for producing noodles, and enzyme preparations useful for modifying noodles.

2. Discussion of the Background

Many foods are collections of a wide variety of components such as starches, proteins, sugars, and lipids, which combine to give a texture to food. The contribution of starches and proteins to texture is particularly large, and the changes with time of starch are considered to be particularly important.

Leaving a gelatinized starch at ordinary temperature or low temperature causes the moisture to separate and the starch hardens. This phenomenon is called retrogradation, and many studies have been made of this phenomenon. Generally, retrogradation prevention requires maintaining the temperature at 80° C. or greater, making the moisture content to be 15% or less by rapid drying, or maintaining alkaline conditions with a pH of 13 or greater. Known methods of preventing retrogradation generally include methods that add sugars (including glucose, fructose, and liquid sugar), soy proteins, wheat glutens, fatty acid esters, or polysaccharides (including yam and konjac) to starch-containing food. JP-A-59-2664 describes a method that adds a thickener, a surfactant, and the like. However, these methods are not sufficient as they greatly change the taste and have unstable effects.

Methods that add enzymes are also known as a means to prevent retrogradation. For example, JP-A-58-86050 describes a method for improving cooked rice, whereby milled rice is cooked with an enzyme such as amylase, protease, and lipase mixed with a common salt and cyclodextrin. JP-A-60-199355 describes a retrogradation preventing method for cooked rice, whereby an aqueous solution of glycosylated amylase (β-amylase, glucoamylase) is sprayed over cooked rice.

There are many findings in texture improving methods for noodles, a variation of starch-containing foods. Specifically, protein materials (including vital gluten, soy protein, egg white, whole egg, and casein) and starches (including various starches, polysaccharides, and emulsifiers) are added to improve the texture of boiled noodles (see JP-A-2-117353). In another method, a short-time, high-temperature treatment is performed in retort sterilization to maintain texture (see JP-A-2-186954). Further, methods are known that use transglutaminase to improve texture (see JP-A-2-286054, JP-A-6-14733). In these methods, an inter- and intra-protein network structure is formed in the noodles by the action of transglutaminase to prevent moisture homogenization in the noodles, making it possible to maintain the preferred elastic (chewy) texture after boiling. However, the texture is uniform overall, and there is still room for improvement in obtaining a texture with a firm center, or Al dente as it is called (harder inside than outside).

WO2005/096839 describes adding a-glucosidase as an agent for improving the physical properties of starch-containing foods during the kneading of wheat, in order to obtain udon that has improved hardness and strength, and that develops a more firm center with time than that obtained without α-glucosidase. While this technique provides certain effects, there is still room for improvement in physical property immediately after boiling. Recently, there is a report that the combined use of α-glucosidase and transglutaminase in appropriate proportions improves the texture immediately after boiling, and, at the same time, maintains the improved texture over extended time periods (see WO2008/001940). The effect is substantial, but is still limited in the sense that strong elasticity cannot be realized in good balance with the preferred texture.

Concerning the use of glucose oxidase for noodles, there is a report that the use of glucose oxidase in combination with amylase and glucoamylase improves chewiness (see JP-A-6-296467). However, the publication does not describe using glucose oxidase with a-glucosidase or transglutaminase. There is also a report that the combined use of glucose oxidase and glucose suppresses mottling, but lowers the texture (see JP-A-11-137197). In another report, glucose oxidase is described as being capable of improving shelf life (Food and Agricultural Materials Inspection Center, Examination Research Report No. 19, p. 95-101); however, there is no description concerning texture improvement. JP-A-2000-60431 discloses a method that uses transglutaminase and glucose oxidase in combination to improve a noodle texture, including the pleasant feel of a noodle through the throat, and firmness. Though this method is highly effective, there are still limits in obtaining a noodle texture that satisfies both “stickiness” and strong “elasticity”.

Thus, there remains a need for methods to reduce retrogradation in noodles.

### SUMMARY OF THE INVENTION

Accordingly, it is one object of the present invention to provide novel methods or producing noodles having improved physical properties and improved taste.

It is another object of the present invention to provide novel enzyme preparations for modifying noodles.

It is another object of the present invention to provide novel methods that improve the qualities (taste and physical properties) of noodles immediately after the production kneading material such as cereal flour, and that suppresses time-dependent quality deterioration as might occur during the producing steps, and during the course of distribution after the production.

It is another object of the present invention to provide novel methods for improving the manufactuability of noodles. More specifically, the invention provides a method for producing noodles with a texture having, for example, both “stickiness” and strong “elasticity”, which cannot be obtained with the sole or combined use of α-glucosidase and transglutaminase alone. Note that “stickiness” is the feel of a noodle clinging to the teeth upon chewing, and “elasticity” is the stress exerted back from chewing, specifically the extent of resilience.

These and other objects, which will become apparent during the following detailed description, have been achieved by the inventors' discovery that contacting the flour used to make noodles, at any time during the noodle production with either:

(1) α-glucosidase and glucose oxidase; or (2) α-glucosidase, glucose oxidase, and transglutaminase, affords noodles with improved properties.

Specifically, the present invention provides the following.
(1) A method for producing a noodle with α-glucosidase and glucose oxidase, wherein the α-glucosidase is used in 1.5 to 300,000 U per gram of a raw material cereal flour, and wherein the glucose oxidase is used in 0.002 to 500 U per gram of the raw material cereal flour and in 0.00003 to 30 U per unit of the α-glucosidase.
(2) The method according to (1), wherein the α-glucosidase is used in 3 to 15,000 U per gram of the raw material cereal flour, and wherein the glucose oxidase is used in 0.005 to 50 U per gram of the raw material cereal flour.
(3) The method according to (1), wherein the glucose oxidase is used in 0.00006 to 3 U per unit of the α-glucosidase.
(4) The method according to (1), wherein transglutaminase is further used.
(5) The method according to (4), wherein the transglutaminase is used in 0.0001 to 100 U per gram of the raw material cereal flour.
(6) The method according to (4), wherein the transglutaminase is used in 0.0001 to 10 U per gram of the raw material cereal flour.
(7) The method according to (4), wherein the transglutaminase is used in 0.0000001 to 1 U per unit of the α-glucosidase.
(8) The method according to (4), wherein the glucose oxidase is used in 0.00006 to 3 U per unit of the α-glucosidase, and wherein the transglutaminase is added in 0.000001 to 0.1 U per unit of the α-glucosidase.
(9) The method according to (1), wherein the noodle is any one of pasta, udon, Chinese-style noodle, stir-fried noodle, and buckwheat noodle.
(10) An enzyme preparation for modifying a noodle with α-glucosidase and glucose oxidase contained as active ingredients, wherein the content of the glucose oxidase is 0.00003 to 30 U per unit of the α-glucosidase.
(11) The enzyme preparation according to (10), wherein the content of the glucose oxidase is 0.00006 to 3 U per unit of the α-glucosidase.
(12) The enzyme preparation according to (10), further containing transglutaminase as an active ingredient.
(13) The enzyme preparation according to (12), wherein the content of the transglutaminase is 0.0000001 to 1 U per unit of the α-glucosidase.
(14) The enzyme preparation according to (12), wherein the content of the transglutaminase is 0.000001 to 0.1 U per unit of the α-glucosidase.

The present invention can improve the qualities of noodles. Specifically, the invention can produce noodles that have both “stickiness” and strong “elasticity”, and can suppress the time-dependent deterioration of noodle quality.

### DETAILED DESCRIPTION OF THE PREFERRED EMBODIMENTS

The noodle producing methods and the enzyme preparations for modifying noodles according to the present invention use α-glucosidase and glucose oxidase, or α-glucosidase, glucose oxidase, and transglutaminase.

The α-glucosidase of the present invention is an enzyme that hydrolyzes the non-reducing terminal α-1,4-glucoside bond to produce α-glucose. Preferably, the α-glucosidase is transglucosidase that has the glycosyl transfer activity to convert the α-1,4 bond to α-1,6 bond. An example of the α-glucosidase is “Transglucosidase L Amano”, commercially available from Amano Enzyme Inc.

The glucose oxidase of the present invention is an oxidase that catalyzes the reaction producing gluconic acid and hydrogen peroxide using glucose, oxygen, and water as the substrates. The hydrogen peroxide produced by this reaction is believed to promote the formation of SS bonds (disulfide bonds) by the oxidation of the SH groups in the protein, and to form a crosslinked structure in the protein. Glucose oxidases of various origins are known, including those from microorganisms and plants. The enzyme used in the present invention may be of any origin, provided that it has the foregoing activity. Further, the enzyme may be a recombinant enzyme. The glucose oxidase of microorganism origin, commercially available under the trade name “Sumizyme PGO” from Shin-Nihon Chemical Co. is one example of such an enzyme. As in many products commercially available as mixtures of glucose oxidase and catalase preparations, the enzyme may be a mixture with other preparations, provided that it has glucose oxidase activity.

The transglutaminase of the present invention refers to the enzyme that has the activity to catalyze the acyl transfer reaction that uses the glutamine residue and lysine residue in the protein or peptide as the donor and the receptor, respectively. Various such enzymes of different origins are known, including those from mammals, fish, and microorganisms. The enzyme used in the present invention may be of any origin, provided that the enzyme has the foregoing activity. Further, the enzyme may be a genetically recombinant enzyme. The transglutaminase of microorganism origin commercially available from Ajinomoto Co., Inc. under the trade name “Activa” TG is one example of such an enzyme.

Among a wide range of noodles available, the invention is considered particularly effective, from the standpoint of such factors as market size and needs, for noodles such as udon, pasta, buckwheat noodle, Chinese-style noodle, stir-fried noodles, and instant noodles produced through a frying step and a drying step, and for the wrapping of jiaozi and shumai.

In the production of noodles (including the wrapping of jiaozi and shumai), the raw material cereal flour such as wheat flour may be acted upon by the α-glucosidase and glucose oxidase, or by the α-glucosidase, glucose oxidase, and transglutaminase at any stage of noodle producing steps in the noodle production using these enzymes. Specifically, the enzymes may be added at the time of mixing the raw materials, or may be sprinkled after mixing. The order in which noodles are acted upon by the transglutaminase, α-glucosidase, and glucose oxidase is not particularly limited, and the enzymes may be allowed to act after one or two of these enzymes have acted first, before the remaining enzyme(s) exhibits its activity.

Preferably, the three enzymes are allowed to act at the same time. The enzymes may be used in combination with other enzymes or substances (sugars such as dextrin, starch, and processed starch; seasonings such as meat extracts; proteins such as plant protein, gluten, egg white, gelatin, and casein; protein hydrolysate; protein partial hydrolysate; emulsifiers; chelating agents such as citrates and polyphosphates; reducing agents such as glutathione and cysteine; and other food additives such as alginic acid, kansui, dye, acidulant, and flavoring ingredient).

Examples of the raw material cereal flour include wheat flour, rice flour, barley flour, and rye flour. The wheat flour used may be of any variety, including, for example, hard flour, semi-hard flour, all-purpose flour, weak flour, and durum semolina flour. Further, the raw material cereal flour may be used by being mixed with a non-wheat cereal flour, such as rice flour, and starch (including processed starch).

In the present invention, the α-glucosidase is added in an appropriate range of 1.5 U or more, preferably 1.5 to 300,000 U, more preferably 3 to 15,000 U in terms of enzyme activity per gram of the raw material cereal flour. Note that 1 U (unit) of α-glucosidase enzyme activity is defined as the amount of the enzyme that produces 1 μg of glucose in 2.5 ml of a reaction liquid when 0.5 ml of an enzyme solution is allowed to act at 40° C. for 60 minutes upon being added to a mixture containing 1 ml of 1 mM a-methyl-D-glucoside and 1 ml of 0.02 M acetate buffer (pH 5.0).

In the present invention, the glucose oxidase is added in an appropriate range of 0.001 U or more, preferably 0.002 to 500 U, more preferably 0.005 to 50 U in terms of enzyme activity per gram of the raw material cereal flour. Further, it is desirable that the glucose oxidase be added in 0.00003 to 30 U, preferably 0.00006 to 3 U per unit of α-glucosidase. Note that the enzyme activity of the glucose oxidase is quantified as follows. The glucose oxidase is allowed to act on the substrate glucose in the presence of oxygen to produce hydrogen peroxide, which is then acted upon by peroxidase in the presence of aminoantipyrine and phenol to produce a quinonimine dye. The color of the quinonimine dye is then measured and quantified at wavelength 500 nm. The amount of enzyme required to oxidize 1 μmol of glucose in 1 minute is defined as 1 U (unit).

In the present invention, the transglutaminase is add in an appropriate range of 0.0001 to 100 U, preferably 0.0001 to 10 U in terms of enzyme activity per gram of the cereal flour. Further, it is desirable that the transglutaminase be added in 0.0000001 to 1 U, preferably 0.000001 to 0.1 U per unit of a-glucosidase. Note that the enzyme activity of the transglutaminase is measured as follows. The hydroxamic acid produced by a reaction using benzyloxycarbonyl-L-glutaminyl-glycine and hydroxylamine as the substrates is used to form an iron complex in the presence of trichloroacetic acid, and the absorbance at 525 nm is measured. The amount of the hydroxamic acid is then determined from a standard curve, and the activity is calculated. The amount of enzyme that produces 1 μmol of hydroxamic acid in 1 minute at 37° C., pH 6.0 is defined as 1 U (unit).

Again, the amounts of enzymes added to produce noodles by the actions of α-glucosidase and glucose oxidase, or by the actions of α-glucosidase, glucose oxidase, and transglutaminase are such that the glucose oxidase is used in an appropriate range of 0.00003 to 30 U, preferably 0.00006 to 3 U in terms of enzyme activity (units) per unit of α-glucosidase, and that the transglutaminase is used in an appropriate range of 0.0000001 to 1 U, preferably 0.000001 to 0.1 U in terms of enzyme activity per unit of a-glucosidase. For pasta, the glucose oxidase is added particularly preferably in an amount of 0.0006 to 3 U per unit of α-glucosidase, and the transglutaminase is added particularly preferably in an amount of 0.000001 to 0.1 U per unit of α-glucosidase. For udon, the glucose oxidase is added particularly preferably in an amount of 0.00006 to 0.3 U per unit of α-glucosidase, and the transglutaminase is added particularly preferably in an amount of 0.000001 to 0.1 U per unit of α-glucosidase. With the foregoing ranges of the addition ratio of the enzymes, a desirable texture that satisfies both stickiness and strong elasticity can be obtained, and the time-dependent quality deterioration of the product noodles can be suppressed. Further, the turbidity of the cooking liquid from boiling can be reduced.

The reaction time of each enzyme is not particularly limited, as long as the enzyme can act on the substrate substance, and may be very brief or long. In practice, the preferred action time is from 5 minutes to 24 hours. The reaction temperature is not particularly limited either, as long as the enzyme can maintain its activity. In practice, the preferred temperature of action is from 0 to 80° C. In other words, the ordinary noodle manufacturing steps provide a sufficient reaction time.

The enzyme preparation for modifying noodles can be obtained from mixtures of α-glucosidase, glucose oxidase, and transglutaminase with bulking agents such as dextrin, starch, and processed starch; seasonings such as meat extract; proteins such as plant protein, gluten, egg white, gelatin, and casein; protein hydrolysate; protein partial hydrolysate, emulsifiers, chelating agents such as citrates and polyphosphates; reducing agents such as glutathione and cysteine; and other food additives such as alginic acid, kansui, dye, acidulant, and flavoring ingredient. The enzyme preparation of the present invention may be in the form of a liquid, a paste, a granule, or a powder. The amount of each enzyme mixed in the enzyme preparation is more than 0% and less than 100%. The mixed amount may be 0% for transglutaminase.

Other features of the invention will become apparent in the course of the following descriptions of exemplary embodiments which are given for illustration of the invention and are not intended to be limiting thereof.

### EXAMPLES

### Example 1

The α-glucosidase preparation “Transglucosidase L” (Amano Enzyme Inc.; hereinafter “AG”), the transglutaminase preparation “Activa” TG (Ajinomoto Co., Inc.; hereinafter, “TG”), and the glucose oxidase preparation Sumizyme PGO (Shin-Nihon Chemical Co. hereinafter, “GO”) were added to 2 kg of duram flour DF (Nisshin Flour Milling Inc), and thoroughly mixed. Test groups are shown in Table 1. After adding 540 g of tap water, the raw material mixture was kneaded for 15 minutes with a kneader “Vacuum mixer VU-2” (kuba Tekkosho) with the kneader speed set to 100. After kneading, noodles were made by extruding the mixture through a 1.8-mm long pasta dice using a pasta machine (vacuum extruder FPV-2; Nippun Engineering Co., Ltd.). The extruded strands were then dried with a drier (constant temperature and humidity vessel LH21-13P; Nagano Science) to obtain dry pasta. The dry pasta was boiled in boiling water for 9 minutes, refrigerated for 24 hours, and heated with a microwave before performing sensory evaluations. The sensory evaluation was done by four panelists for stickiness, elasticity, hardness, firm center, and ease of bite, using the scales from −2 to 2 with a 0 score for control group. The results are shown in Table 1. Note that “stickiness” is the feel of a noodle clinging to the teeth upon chewing, “elasticity” is the stress exerted back from chewing, specifically the extent of resilience, “hardness” is the stress felt upon biting, “firm center” is the Al dente-like texture being harder inside than outside with a hardness gradient from outside toward the center of the strand, and “ease of bite” is the measure of how easily the teeth bite into and cut the noodles. The scales are 0.5=difference present, 1=notable difference, 2=very notable difference. Because of the importance of having both stickiness and strong elasticity for the texture of pasta, the following denotation was used for the test groups:

“*”: Stickiness (scores higher than 0)

“*”: Notable elasticity (scores of 1 and higher)

“*”: Elasticity with the scores of 1.75 and higher

The noodles were deemed to have both stickiness and strong elasticity when the score for stickiness was higher than 0, and when the score for elasticity was 1 or higher, as above.

TABLE 1

| | | | | Enzyme activity | | | | | | | | |
|---|---|---|---|---|---|---|---|---|---|---|---|---|
| Test | U/g (Cereal flour) | | | ratio | | Sensory evaluation results | | | | | | |
| group | AG | TG | GO | GO/AG | TG/AG | Stickiness | | Elasticity | | Hardness | Firm center | Ease of bite |
| Control | 0 | 0 | 0 | 0 | 0 | 0 | | 0 | | 0 | 0 | 0 |
| 1 | 0 | 0 | 0.924 | 0 | 0 | 0 | | 1.25 | * | 0.75 | 0.25 | 1 |
| 2 | 0 | 0.633 | 0 | 0 | 0 | −0.5 | | 0.25 | | 2 | 0 | 1 |
| 3 | 343.8 | 0 | 0 | 0 | 0 | 0.25 | * | 0 | | −0.25 | 0.25 | −0.5 |
| 4 | 137.5 | 0 | 0.693 | 0.005 | 0 | 1 | * | 1 | * | 0.1 | 0.75 | 0.25 |
| 5 | 68.8 | 0 | 0.924 | 0.0134 | 0 | 2 | * | 1.25 | * | 0.25 | 1.25 | 0.75 |
| 6 | 68.8 | 0 | 1.386 | 0.0201 | 0 | 0.75 | * | 1.5 | * | 0.5 | 0.75 | 1.25 |
| 7 | 68.8 | 0.253 | 0.924 | 0.0134 | 0.0037 | 0.5 | * | 2 | ** | 1.75 | 0.25 | 1.5 |
| 8 | 68.8 | 0.127 | 1.155 | 0.0168 | 0.0018 | 0.75 | * | 1.75 | ** | 1.5 | 1 | 1.25 |

As shown in Table 1, GO imparted strong elasticity, but did not impart any stickiness important for providing a desirable texture for pasta. While AG imparted stickiness, hardly any elasticity was imparted, and hardness lowered. Both stickiness and elasticity were imparted in test groups that used AG and GO in combination, and even stronger elasticity was imparted in test groups that also used TG in combination with AG and GO. The results thus demonstrated that pasta with a desirable texture having both stickiness and strong elasticity could be produced with the combined use of AG and GO, or with the combined use of AG, TG, and GO.

### Example 2

AG, TG, and GO were added to 750 g of the all-purpose flour Suzume (Nisshin Flour Milling Inc.), 250 g of the processed starch Ajisai (Matsutani Chemical Industry Co., Ltd.), and 20 g of the wheat gluten A-Glu G (Glico Foods, Co., Ltd.), and mixed for 1 minute at 100 rpm with a 2-kg vacuum kneader (Ohtake Noodle Machine Mfg., Co., Ltd.). The test groups are shown in Table 2. In Table 2, the cereal flour is all-purpose flour, and does not include processed starch. A 5° C. brine prepared by adding 30 g of a common salt to 410 g of tap water was added to the total amount of the raw material mixture, and kneaded with a kneader for 5 minutes (100 rpm for 2 minutes, 50 rpm for 3 minutes). After being kneaded, the mixture was processed into a sheet with noodle making machines (small coarse noodle sheeter, small continuous rolling machine; Tom), combined, and press-rolled. The sheet was matured for 1 hour at room temperature, and cut with a #10 cutting blade. The strands were immediately frozen to obtain frozen uncooked udon. The frozen uncooked udon was boiled in boiling water for 7.5 minutes, and refrigerated for 24 hours before performing sensory evaluations. The sensory evaluation was done by four panelists for stickiness, elasticity, hardness, firm center, and stickiness, using the scales from −2 to 2 with a 0 score for control group. The results are shown in Table 2. Note that “stickiness” is the feel of a noodle clinging to the teeth upon chewing, “elasticity” is the stress exerted back from chewing, specifically the extent of resilience, “hardness” is the stress felt upon biting, “firm center” is the Al dente-like texture being harder inside than outside with a hardness gradient from outside toward the center of the strand, and “stickiness” is the measure of adhesion to the teeth after chewing. The scales are 0.5=difference present, 1=notable difference, 2=very notable difference. Because of the importance of having both stickiness and strong elasticity for the texture of udon, the following denotation was used for the test groups:

“*”: Stickiness (scores higher than 0)

“*”: Notable elasticity (scores of 1 and higher)

TABLE 2

| | | | | Enzyme activity | | | | | | | | |
|---|---|---|---|---|---|---|---|---|---|---|---|---|
| Test | U/g (Cereal flour) | | | ratio | | Sensory evaluation results | | | | | | |
| group | AG | TG | GO | GO/AG | TG/AG | Stickiness | | Elasticity | | Hardness | Firm center | Ease of bite |
| Control | | | | | | 0 | | 0 | | 0 | 0 | 0 |
| 1 | | | 0.700 | | | 0 | | 2 | * | 1.5 | 0 | 0 |
| 2 | | 0.383 | | | | −1 | | 0.5 | | 2 | 0 | −1 |
| 3 | 208.3 | | | | | 0.25 | * | 0.25 | | −0.5 | 0.25 | 2 |
| 4 | 166.7 | 0.077 | | | 0.0005 | 1.75 | * | 0.5 | | 0.5 | 1.75 | 1.75 |
| 5 | 166.7 | 0.061 | 0.028 | 0.0002 | 0.0004 | 2 | * | 1 | * | 0.5 | 2 | 1.75 |
| 6 | 166.7 | 0.046 | 0.056 | 0.0003 | 0.0003 | 1.5 | * | 1 | * | 0.75 | 1.5 | 1.5 |
| 7 | 166.7 | 0.031 | 0.084 | 0.0005 | 0.0002 | 1.25 | * | 1.25 | * | 0.75 | 1.25 | 1.25 |
| 8 | 166.7 | 0.015 | 0.112 | 0.0007 | 0.0001 | 1 | * | 1.25 | * | 0.75 | 1 | 1 |
| 9 | 166.7 | | 0.140 | 0.0008 | | 1 | * | 1.25 | * | 0.75 | 0.75 | 0.75 |
| 10 | 166.7 | | 0.056 | 0.0003 | | 1.25 | * | 1 | * | 0.25 | 1 | 1.25 |
| 11 | 83.3 | | 0.420 | 0.0050 | | 0.5 | * | 1.75 | * | 1.25 | 0.5 | 0.5 |
| 12 | 41.7 | | 0.560 | 0.0134 | | 0.25 | * | 1.75 | * | 1.25 | 0.5 | 0.25 |
| 13 | 41.7 | 0.153 | 0.560 | 0.0134 | 0.0037 | 0.25 | * | 2 | * | 2 | 0.25 | 0.25 |
| 14 | 41.7 | 0.153 | 0.280 | 0.0067 | 0.0037 | 0.5 | * | 1.75 | * | 1.75 | 0.25 | 0.5 |
| 15 | 41.7 | 0.153 | 0.140 | 0.0034 | 0.0037 | 0.5 | * | 1.25 | * | 1.25 | 0.5 | 0.5 |
| 16 | 83.3 | 0.077 | 0.560 | 0.0067 | 0.0009 | 0.5 | * | 1.75 | * | 1.75 | 0.25 | 0.5 |
| 17 | 83.3 | 0.077 | 0.280 | 0.0034 | 0.0009 | 0.75 | * | 1.5 | * | 1.5 | 0.5 | 0.75 |
| 18 | 83.3 | 0.077 | 0.140 | 0.0017 | 0.0009 | 0.75 | * | 1.25 | * | 1 | 1 | 0.75 |
| 19 | 166.7 | 0.077 | 0.140 | 0.0008 | 0.0005 | 1 | * | 1.5 | * | 1 | 0.75 | 1.25 |
| 20 | 20.8 | 0.077 | 0.280 | 0.0134 | 0.0037 | 0.25 | * | 1.5 | * | 1.5 | 0.25 | 0.25 |

As shown in Table 2, GO imparted strong elasticity, but did not impart any stickiness important for providing a desirable texture for udon. While AG imparted stickiness, sufficient levels of elasticity were not imparted, and hardness lowered. In test groups in which AG and TG were used in combination, the stickiness was notable, and the elasticity was greater than that obtained with the sole use of AG. However, the levels of elasticity were still not sufficient. Both stickiness and strong elasticity were imparted in test groups in which AG and GO, or AG, TG, and GO were used in combination. The results demonstrated that udon with a desirable texture having both stickiness and strong elasticity could be produced with the combined use of AG and GO, or with the combined use of AG, TG, and GO.

### Example 3

Udon was produced in the test groups of Table 3 from the same raw materials used in Example 2 according to the methods of Example 2, and sensory evaluations were performed. The sensory evaluations were performed for the desirable udon texture satisfying both stickiness and strong elasticity, according to the following criteria: “x”=undesirable, “Δ”=marginally desirable, “O”=desirable, and “OO”=very desirable.

As can be seen in test groups 1 to 9 in Table 3, desirable textures with stickiness and strong elasticity, which means that the results were “Δ” or better were obtained with 0.00003 to 30 U of GO added per unit of AG. Similarly, in test groups 10 to 18, desirable textures were obtained with 1 U or less of TG added per unit of AG. It was also confirmed that desirable textures could be obtained with at least 0.0000001 U. Desirable textures were also obtained in test groups 19 to 27 with 1.5 to 300000 U of AG activity per gram of the raw material cereal flour, and with 0.002 to 500 U of GO activity per gram of the raw material cereal flour. The texture was also desirable in test groups 28 to 35, in which the TG activity per gram of the raw material cereal flour was 100 U or less. It was also confirmed that desirable textures were obtained with at least 0.0001 U. Desirable effects were not obtained in the test groups out of the foregoing ranges, in which the effect of each enzyme was either too weak or too strong, disrupting the overall texture balance.

The results confirmed that desirable textures could be obtained with the combined use of AG and GO, or with the combined use of AG, TG, and GO in noodles, when the amount of AG added per gram of the raw material cereal flour is 1.5 to 300000 U, the amount of GO added per gram of the raw material cereal flour is 0.002 to 500 U, and the amount of TG added per gram of the raw material cereal flour is 0.0001 to 100 U and when the amount of GO added per unit of AG is 0.00003 to 30 U and the amount of TG added per unit of AG is 0.0000001 to 1 U.

TABLE 3

| Test | U/g (Cereal flour) | | | Enzyme activity ratio | | Sensory evaluation |
|---|---|---|---|---|---|---|
| group | AG | TG | GO | GO/AG | TG/AG | results |
| Control | 0 | 0 | 0 | 0 | 0 | X |
| 1 | 167 | 0 | 0.0005 | 0.000003 | 0 | X |
| 2 | 167 | 0 | 0.005 | 0.00003 | 0 | Δ |
| 3 | 167 | 0 | 0.05 | 0.0003 | 0 | ◯ |
| 4 | 167 | 0 | 0.5 | 0.003 | 0 | ◯◯ |
| 5 | 17 | 0 | 0.5 | 0.03 | 0 | ◯◯ |
| 6 | 1.7 | 0 | 0.5 | 0.3 | 0 | ◯ |
| 7 | 1.7 | 0 | 5 | 3 | 0 | ◯ |
| 8 | 1.7 | 0 | 50 | 30 | 0 | Δ |
| 9 | 1.7 | 0 | 500 | 300 | 0 | X |
| 10 | 16667 | 0.0015 | 50 | 0.003 | 0.0000001 | ◯ |
| 11 | 1667 | 0.0015 | 5 | 0.003 | 0.000001 | ◯ |
| 12 | 167 | 0.0015 | 0.5 | 0.003 | 0.00001 | ◯ |
| 13 | 167 | 0.015 | 0.5 | 0.003 | 0.0001 | ◯◯ |
| 14 | 167 | 0.15 | 0.5 | 0.003 | 0.001 | ◯◯ |
| 15 | 167 | 1.5 | 0.5 | 0.003 | 0.01 | ◯◯ |
| 16 | 167 | 15 | 0.5 | 0.003 | 0.1 | ◯ |
| 17 | 17 | 15 | 0.05 | 0.003 | 1 | Δ |
| 18 | 17 | 160 | 0.05 | 0.003 | 10 | X |
| 19 | 600000 | 0 | 1000 | 0.0017 | 0 | X |
| 20 | 300000 | 0 | 500 | 0.0017 | 0 | Δ |
| 21 | 30000 | 0 | 50 | 0.0017 | 0 | ◯ |
| 22 | 3000 | 0 | 5 | 0.0017 | 0 | ◯ |
| 23 | 300 | 0 | 0.5 | 0.0017 | 0 | ◯◯ |
| 24 | 30 | 0 | 0.05 | 0.0017 | 0 | ◯ |
| 25 | 3 | 0 | 0.005 | 0.0017 | 0 | ◯ |
| 26 | 1.5 | 0 | 0.002 | 0.0017 | 0 | Δ |
| 27 | 0.3 | 0 | 0.0005 | 0.0017 | 0 | X |
| 28 | 600000 | 200 | 1000 | 0.0017 | 0.0003 | X |
| 29 | 300000 | 100 | 500 | 0.0017 | 0.0003 | Δ |
| 30 | 30000 | 10 | 50 | 0.0017 | 0.0003 | ◯ |
| 31 | 3000 | 1 | 5 | 0.0017 | 0.0003 | ◯ |
| 32 | 300 | 0.1 | 0.5 | 0.0017 | 0.0003 | ◯◯ |
| 33 | 30 | 0.01 | 0.05 | 0.0017 | 0.0003 | ◯ |
| 34 | 3 | 0.001 | 0.005 | 0.0017 | 0.0003 | ◯ |
| 35 | 3 | 0.0001 | 0.005 | 0.0017 | 0.00003 | ◯ |

### Example 4

AG, TG, and GO were added to 1,000 g of the all-purpose flour Shirotsubaki (Nisshin Flour Milling Inc.), and 1 g of the gardenia dye Yellow Color TH-G (T. Hasegawa Co., Ltd.), and mixed for 1 minute at 100 rpm with a 2kg-vacuum kneader (Ohtake Noodle Machine Mfg., Co., Ltd.). The test groups are shown in Table 4. A 5° C. solution prepared by adding 5 g of a common salt and 10 g of an alkaline preparation (powdery kansui A; Nippon-Colloid) to 420 g of tap water was then added to the total amount of the raw material mixture, and the mixture was kneaded with a kneader for 3.5 minutes (100 rpm for 2 minutes, 50 rpm for 1.5 minutes). After being kneaded, the mixture was formed into a sheet using noodle making machines (small coarse noodle sheeter, small continuous rolling machine; Tom), combined, and press-rolled. The sheet was matured for 1 hour at room temperature, and cut with a #18 cutting blade. The strands were immediately frozen to obtain frozen uncooked Chinese-style noodles. The frozen Chinese-style noodles were boiled in boiling water for 2.5 minutes, and refrigerated for 24 hours before performing sensory evaluations for cold Chinese-style noodles. The sensory evaluation was done by four panelists for stickiness and elasticity, using the scales from −2 to 2 with a 0 score for control group. The results are shown in Table 4. The scales are 0.5=difference present, 1=notable difference, 2=very notable difference. Because of the importance of having both stickiness and strong elasticity for the texture of Chinese-style noodles, the following denotation was used for the test groups:

“*”: Stickiness (scores higher than 0)

“*”: Notable elasticity (scores of 1 and higher)

TABLE 4

| | U/g (Cereal flour) | | | Enzyme activity ratio | | Sensory evaluation results | | | |
|---|---|---|---|---|---|---|---|---|---|
| Test group | AG | TG | GO | GO/AG | TG/AG | Stickiness | | Elasticity | |
| Control | 0 | 0 | 0 | 0 | 0 | 0 | | 0 | |
| 1 | 208 | 0 | 0 | 0 | 0 | 0.5 | * | 0.25 | |
| 2 | 0 | 0.383 | 0 | 0 | 0 | −1 | | 0.5 | |
| 3 | 0 | 0 | 0.7 | 0 | 0 | 0 | | 2 | * |
| 4 | 188 | 0 | 0.63 | 0.0034 | 0 | 1.75 | * | 1 | * |
| 5 | 167 | 0 | 0.14 | 0.0008 | 0 | 1.5 | * | 1.25 | * |
| 6 | 83 | 0 | 0.42 | 0.005 | 0 | 1 | * | 1.5 | * |
| 7 | 42 | 0 | 0.56 | 0.0134 | 0 | 0.75 | * | 1.75 | * |
| 8 | 42 | 0.153 | 0.56 | 0.0134 | 0.0037 | 0.5 | * | 2 | * |
| 9 | 83 | 0.077 | 0.56 | 0.0067 | 0.0009 | 0.75 | * | 1.75 | * |
| 10 | 175 | 0.077 | 0 | 0 | 0.0004 | 1 | * | 0.5 | |

As shown in Table 4, addition of only GO imparted strong elasticity by the action of GO, but did not impart any stickiness important for providing a desirable texture for Chinese-style noodles. The addition of only AG imparted stickiness by the action of AG, but did not impart sufficient levels of elasticity. Both stickiness and strong elasticity were imparted in test groups in which AG and GO, or AG, TG, and GO were used in combination. The results demonstrated that Chinese-style noodles with a desirable texture having both stickiness and strong elasticity could be produced with the combined use of AG and GO, or with the combined use of AG, TG, and GO.

### Example 5

Frozen uncooked Chinese-style noodles were obtained in the test groups of Table 5 according to the methods of Example 4. The frozen uncooked Chinese-style noodles were gently loosened, steamed for 7 minutes, and stir-fried for 30 seconds with a source to obtain stir-fried noodles. The stir-fried noodles were refrigerated for 24 hours, and heated in a microwave before performing sensory evaluations. The sensory evaluation was done by four panelists for stickiness and elasticity, using the scales from −2 to 2 with a 0 score for control group. The results are shown in Table 5. The scales are 0.5=difference present, 1=notable difference, 2=very notable difference. Because of the importance of having both stickiness and strong elasticity for the texture of stir-fried noodles, the following denotation was used for the test groups:

“*”: Stickiness (scores higher than 0)

“*”: Notable elasticity (scores of 1 and higher)

As shown in Table 5, the addition of only GO imparted strong elasticity by the action of GO, but did not impart any stickiness important for providing a desirable texture for stir-fried noodles. The addition of only AG imparted stickiness and elasticity by the action of AG, but the levels of elasticity were slightly weak. Both stickiness and strong elasticity were imparted in test groups in which AG and GO, or AG, TG, and GO were used in combination. The results demonstrated that stir-fried noodles with a desirable texture having both stickiness and strong elasticity could be produced with the combined use of AG and GO, or with the combined use of AG, TG, and GO.

TABLE 5

| | U/g (Cereal flour) | | | Enzyme activity ratio | | Sensory evaluation results | | | |
|---|---|---|---|---|---|---|---|---|---|
| Test group | AG | TG | GO | GO/AG | TG/AG | Stickiness | | Elasticity | |
| Control | 0 | 0 | 0 | 0 | 0 | 0 | | 0 | |
| 1 | 208 | 0 | 0 | 0 | 0 | 0.5 | * | 0.25 | |
| 2 | 0 | 0.383 | 0 | 0 | 0 | −1 | | 0.25 | |
| 3 | 0 | 0 | 0.7 | 0 | 0 | 0 | | 2 | * |
| 4 | 188 | 0 | 0.63 | 0.0034 | 0 | 2 | * | 1 | * |
| 5 | 167 | 0 | 0.14 | 0.0008 | 0 | 2 | * | 1.25 | * |
| 6 | 83 | 0 | 0.42 | 0.005 | 0 | 1.5 | * | 1.5 | * |
| 7 | 42 | 0 | 0.56 | 0.0134 | 0 | 1 | * | 1.75 | * |
| 8 | 42 | 0.153 | 0.56 | 0.0134 | 0.0037 | 0.75 | * | 2 | * |
| 9 | 83 | 0.077 | 0.56 | 0.0067 | 0.0009 | 1 | * | 1.75 | * |
| 10 | 175 | 0.077 | 0 | 0 | 0.0004 | 1.25 | * | 0.75 | |

### Example 6

AG, TG, and GO were added to 500 g of buckwheat flour Heiwa (Hokuto Flour Milling Co., Ltd.) and 500 g of hard flour Seikei (Nissin Seifun), and mixed for 1 minute at 100 rpm with a 2-kg vacuum kneader (Ohtake Noodle Machine Mfg., Co., Ltd.). The test groups are shown in Table 6. In Table 6, the cereal flour means both buckwheat flour and hard flour. A 5° C. brine prepared by adding 15 g of a common salt to 350 g of tap water was added to the total amount of the raw material mixture, and the mixture was kneaded with a kneader for 5 minutes (100 rpm for 2 minutes, 50 rpm for 3 minutes). After being kneaded, the mixture was formed into a sheet with noodle making machines (small coarse noodle sheeter, small continuous rolling machine; Tom), combined, and press-rolled. The sheet was matured for 1 hour at room temperature, and cut with a #18 cutting blade. The strands were immediately frozen to obtain frozen uncooked buckwheat noodle. The frozen uncooked buckwheat noodle was boiled in boiling water for 2.5 minutes, and refrigerated for 24 hours before performing sensory evaluations. The sensory evaluation was done by four panelists for stickiness and elasticity, using the scales from −2 to 2 with a 0 score for control group. The results are shown in Table 6. Despite that important textures for buckwheat noodle generally include hardness, elasticity, and ease of bite, because stickiness and elasticity are considered important in varieties such as country-style buckwheat noodle, the following denotation was used for the test groups in the invention:

“*”: Stickiness (scores higher than 0)

“*”: Notable elasticity (scores of 1 and higher)

TABLE 6

| | U/g (Cereal flour) | | | Enzyme activity ratio | | Sensory evaluation results | | | |
|---|---|---|---|---|---|---|---|---|---|
| Test group | AG | TG | GO | GO/AG | TG/AG | Stickiness | | Elasticity | |
| Control | 0 | 0 | 0 | 0 | 0 | 0 | | 0 | |
| 1 | 208 | 0 | 0 | 0 | 0 | 0.5 | * | 0.25 | |
| 2 | 0 | 0.383 | 0 | 0 | 0 | −1 | | 0.25 | |
| 3 | 0 | 0 | 0.7 | 0 | 0 | 0 | | 2 | * |
| 4 | 42 | 0.153 | 0.56 | 0.0134 | 0.0037 | 0.5 | * | 2 | * |
| 5 | 42 | 0.153 | 0 | 0 | 0.0037 | 0.75 | * | 0.5 | |
| 6 | 42 | 0 | 0.56 | 0.0134 | 0 | 1 | * | 1.75 | * |
| 7 | 0 | 0.153 | 0.56 | 0 | 0 | −0.5 | | 1.5 | * |

As shown in Table 6, the addition of only GO imparted strong elasticity by the action of GO, but did not impart any stickiness. The addition of only AG imparted stickiness and elasticity by the action of AG, but the levels of elasticity were slightly weak. Both stickiness and strong elasticity were imparted in test groups in which AG and GO, or AG, TG, and GO were used in combination. The results demonstrated that buckwheat noodle with a desirable texture having both stickiness and strong elasticity could be produced with the combined use of AG and GO, or with the combined use of AG, TG, and GO.

### Comparative Example.

JP-A-6-296467 describes improving chewiness with the combined use of glucose oxidase, amylase (AM), and glucoamylase (GA). For comparison with the present invention, udon was prepared using the same materials and methods used in Example 2 and a sensory evaluation was done. The sensory evaluation was done by four panelists for stickiness and elasticity, using the scales from −2 to 2 with a 0 score for control group. The results are shown in Table 7. The scales are 0.5=difference present, 1=notable difference, 2=very notable difference. Because of the importance of having both stickiness and strong elasticity for the texture of udon, the following denotation was used for the test groups:

“*”: Stickiness (scores higher than 0)

“*”: Notable elasticity (scores of 1 and higher)

The test groups are shown in Table 7. Note that Amylase AD Amano 1 and Gluczyme AF6 (both available from Amano Enzyme Inc.) were used as α-amylase and glucoamylase, respectively. The amounts of α-amylase, glucoamylase, and GO in the test groups 1 to 3 in Table 7 were set according to Example 1, Comparative Example 2, and Comparative Example 3 of JP-A-6-296467.

TABLE 7

| | | | | | | Enzyme activity | | | | | |
|---|---|---|---|---|---|---|---|---|---|---|---|
| Test | U/g (Cereal flour) | | | | | ratio | | Sensory evaluation results | | | |
| group | AM | GA | GO | AG | TG | GO/AG | TG/AG | Stickiness | | Elasticity | |
| Control | 0 | 0 | 0 | 0 | 0 | 0 | 0 | 0 | | 0 | |
| 1 | 0.5 | 0 | 0 | 0 | 0 | 0 | 0 | 0.25 | * | −0.25 | |
| 2 | 0.4 | 0.01 | 0 | 0 | 0 | 0 | 0 | 0.25 | * | −0.25 | |
| 3 | 0.3 | 0.01 | 0.2 | 0 | 0 | 0 | 0 | 0.75 | * | 0.5 | |
| 4 | 0 | 0 | 0.2 | 0 | 0 | 0 | 0 | 0 | | 2 | * |
| 5 | 0 | 0 | 0.2 | 300 | 0 | 0.0007 | 0 | 1.5 | * | 1.5 | * |
| 6 | 0 | 0 | 0.2 | 300 | 0.1 | 0.0007 | 0.0003 | 1 | * | 2 | * |

As shown in Table 7, the addition of amylase, or the addition of amylase and glucoamylase imparted stickiness; however, these additions tended to weaken elasticity slightly. The strong levels of elasticity as obtained in the present invention could not be obtained with the combined use of amylase, glucoamylase, and GO. The texture obtained from the use of amylase was mainly characterized by heavy, gluey stickiness, largely different from the feel of stickiness, which is the feel of the noodle clinging to the teeth upon chewing, as defined in the present invention. Further, the use of amylase had a pronounced effect on softness, and had the tendency to make the elasticity slightly weak. Additional tests confirmed that this tendency became more pronounced as the reaction time of the enzyme increased in the producing steps. This greatly differs from the present invention. The combined use of GO and AG, or the combined use of GO, AG, and TG provided very desirable textures that satisfied both stickiness and strong elasticity. These results demonstrated that the present invention, capable of imparting stickiness and strong elasticity with the combined use of AG and GO, or AG, GO, and TG without imparting stickiness or softness, is greatly different from JP-A-6-296467, and represents a useful finding.

### INDUSTRIAL APPLICABILITY

The present invention can improve noodle qualities, and is therefore highly useful in the field of food.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for producing a noodle, comprising
(a) contacting flour with α-glucosidase and glucose oxidase; and
(b) producing a noodle from said flour,
wherein:
said contacting is carried out at any time during said producing,
said flour is contacted with said α-glucosidase in an amount of 1.5 to 300,000 U per gram of a raw material cereal flour, and
said flour is contacted with said glucose oxidase in an amount of 0.002 to 500 U per gram of the raw material cereal flour and in an amount of 0.00003 to 30 U per unit of said α-glucosidase.

2. A method according to claim 1, wherein said flour is contacted with said α-glucosidase in an amount of 3 to 15,000 U per gram of the raw material cereal flour, and said flour is contacted with said glucose oxidase in an amount of 0.005 to 50 U per gram of the raw material cereal flour.

3. A method according to claim 1, wherein said flour is contacted with said glucose oxidase in an amount of 0.00006 to 3 U per unit of said α-glucosidase.

4. A method according to claim 1, wherein said flour is further contacted with transglutaminase.

5. A method according to claim 4, wherein said flour is contacted with said transglutaminase in an amount of 0.0001 to 100 U per gram of the raw material cereal flour.

6. A method according to claim 4, wherein said flour is contacted with said transglutaminase in an amount of 0.0001 to 10 U per gram of the raw material cereal flour.

7. A method according to claim 4, wherein said flour is contacted with said transglutaminase in an amount of 0.0000001 to 1 U per unit of said α-glucosidase.

8. A method according to claim 4, wherein said flour is contacted with said glucose oxidase in an amount of 0.00006 to 3 U per unit of said α-glucosidase, and said flour is contacted with said transglutaminase in an amount of 0.000001 to 0.1 U per unit of said α-glucosidase.

9. A method according to claim 1, wherein said noodle is selected from the group consisting of pasta, udon, Chinese-style noodle, stir-fried noodle, and buckwheat noodle.

10. An enzyme preparation, comprising α-glucosidase and glucose oxidase, wherein said glucose oxidase is present in an amount of 0.00003 to 30 U per unit of said α-glucosidase.

11. An enzyme preparation according to claim 10, wherein said glucose oxidase is present in an amount of 0.00006 to 3 U per unit of said α-glucosidase.

12. An enzyme preparation according to claim 10, further comprising transglutaminase.

13. An enzyme preparation according to claim 12, wherein said transglutaminase is present in an amount of 0.0000001 to 1 U per unit of said α-glucosidase.

14. An enzyme preparation according to claim 12, wherein said transglutaminase is present in an amount of 0.000001 to 0.1 U per unit of said α-glucosidase.

15. In a method for producing a noodle, comprising producing a noodle from a flour, the improvement comprising:
(a) contacting said flour with α-glucosidase and glucose oxidase, wherein:
said contacting is carried out at any time during said producing,
said flour is contacted with said α-glucosidase in an amount of 1.5 to 300,000 U per gram of a raw material cereal flour, and
said flour is contacted with said glucose oxidase in an amount of 0.002 to 500 U per gram of the raw material cereal flour and in an amount of 0.00003 to 30 U per unit of the α-glucosidase.
